SEQUENCE LISTING (2 sequences)

SEQ ID NO: 1 - DNA, 50 bp, Artificial Sequence
  16S_V3_F
  misc_feature (42)..(42): n is a or g or c or t/u, unknown, or other.
  misc_feature (46)..(46): w is a or t/u.
tcgtcggcagcgtcagatgtgtataagagacagcctacgggnggcwgcag

SEQ ID NO: 2 - DNA, 55 bp, Artificial Sequence
  16S_V4_R
  misc_feature (41)..(41): h is a or c or t/u.
  misc_feature (42)..(42): v is a or g or c.
gtctcgtgggctcggagatgtgtataagagacaggactachvgggtatctaatcc

(19) United States
(12) Patent Application Publication
(10) Pub. No.: US 2023/0193394 A1
(43) Pub. Date: Jun. 22, 2023

(54) METHOD FOR DIAGNOSING BREAST CANCER VIA MICROBIAL METAGENOMIC ANALYSIS

(71) Applicant: MD HEALTHCARE INC., Seoul (KR)

(72) Inventor: Yoon-Keun KIM, Namyangju-si, Gyeonggi-do (KR)

(73) Assignee: MD HEALTHCARE INC., Seoul (KR)

(21) Appl. No.: 16/474,048

(22) PCT Filed: Dec. 20, 2017
(86) PCT No.: PCT/KR2017/015115

§ 371 Date: Jun. 26, 2019

(30) Foreign Application Priority Data
Dec. 26, 2016 (KR) 10-2016-0179247
Jun. 13, 2017 (KR) 10-2017-0074046

(51) Int. Cl. C12Q 1/6886; C12Q 1/689; C12Q 1/6806

(52) U.S. Cl. CPC C12Q 1/6886; C12Q 1/689; C12Q 1/6806; C12Q 2600/16; C12Q 2600/158

(57) ABSTRACT

A method of diagnosing breast cancer through bacterial and archaeal metagenomic analysis is provided, and more particularly, a method of diagnosing breast cancer by analyzing an increase or decrease in content of extracellular vesicles derived from specific bacteria and archaea through bacterial and archaeal metagenomic analysis using a sample derived from a subject. Extracellular vesicles secreted from bacteria or archaea present in the environment are absorbed into the human body, and thus may directly affect the occurrence of cancer, and it is difficult to diagnose breast cancer early before symptoms occur, and thus efficient treatment therefor is difficult. A risk of developing breast cancer can be predicted through bacterial and archaeal metagenomic analysis using a human body-derived.

### TECHNICAL FIELD

The present invention relates to a method of diagnosing breast cancer through microbial metagenomic analysis, and more particularly, to a method of diagnosing breast cancer by analyzing an increase or decrease in content of extracellular vesicles derived from specific bacteria and archaea through metagenomic analysis of a microorganism such as bacteria, archaea, or the like using a sample derived from a subject.

### BACKGROUND ART

Breast carcinoma collectively refers to all malignant tumors occurring in the breast. Breast cancer is a fatal disease that continues to grow in abnormal breast tissue or spreads to other organs. Although breast cancer has been researched most among all cancers, there is only indefinite knowledge that breast cancer occurs by two factors: an environmental factor and a genetic factor, and there is still no established theory regarding the causes of the onset of breast cancer. However, according to several research results, there is a consensus that several factors are highly correlated and among these, estrogen, which is a female hormone, plays an important role in the development of breast cancer. The risk factors for causes of the onset of breast cancer have been known, and it was reported that, when parents, a sibling, or a daughter in the family has breast cancer, the risk of developing breast cancer himself or herself increases 1.7 times, particularly, 2.4 times in the case of breast cancer occurring prior to menopause, and 9 times in a case in which two or more of the family members have bilateral breast cancer. According to foreign reports, about 10% of breast cancer patients have inherited breast cancer with breast cancer genes (BRCA-1 and BRCA-2). 60% and 85% of individuals with breast cancer genes develop breast cancer prior to 50 years of age and by 70 years of age, respectively, and 65% may simultaneously develop ovarian cancer by 70 years of age.

In addition, long-term activation of reproductive hormones may lead to breast cancer due to carcinogenic mutations of ductal epithelial cells. In particular, it has been known that women having early menarche (before 12 years old) or late menopause (two-fold after 55 years old), women who did not have a child, women who had the first pregnancy after 30 years old, premenopausal women who have no ability to breastfeed, women who have taken contraceptives for 10 years or longer, and women who have received hormone replacement therapy for 10 years or longer to treat facial flushes occurring in menopause or prevent osteoporosis or heart disease have a risk for developing breast cancer. As other environmental factors, it is known to be correlated with drinking, high-fat diets (particularly, polyunsaturated fats such as corn oil, margarine, and the like and saturated fat of beef), overweight postmenopausal women, radiation exposure, and the like. The age at onset of breast cancer is generally higher than that of benign breast disease, and although the average age is 45 years old for Korean women and 55 years old for American women, which is a 10-year difference, the frequency increases with age.

However, despite the fact that breast cancer is most researched among solid cancers, there is still no method of predicting breast cancer using a non-invasive method, and there are many cases in which solid cancers such as breast cancer and the like are detected after the onset thereof, using existing diagnosis methods. Thus, to reduce medical costs and prevent death due to breast cancer, it is efficient to provide a method of predicting the onset of breast cancer and causative factors thereof and preventing the onset of breast cancer in a high-risk group.

Meanwhile, it is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat. The intestinal microbiota is known to play a vital role in human’s physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment, and collectively refers to a total genome of all microbiota in the natural environment in which microorganisms exist and was first used by Jo Handelsman in 1998 (Handelsman et al., 1998 Chem. Biol. 5, R245-249). Recently, the bacterial composition of human microbiota has been listed using a method based on 16 s ribosomal RNA (16 s rRNA) base sequences, and 16 s rDNA base sequences, which are genes of 16 s ribosomal RNA, are analyzed using a next generation sequencing (NGS) platform.

In the onset of breast cancer, however, identification of causative factors of breast cancer through metagenomic analysis of microbe-derived vesicles isolated from a human-derived substance, such as blood, urine or the like, and a method of predicting breast cancer have never been reported.

### DISCLOSURE

### Technical Problem

To diagnose breast cancer, the inventors of the present invention extracted DNA from bacteria- and archaea-derived extracellular vesicles using serum, which is a subject-derived sample, and performed metagenomic analysis on the extracted DNA, and, as a result, identified bacteria-derived extracellular vesicles capable of acting as a causative factor of breast cancer, thus completing the present invention based on these findings.

Therefore, it is an object of the present invention to provide a method of providing information for breast cancer diagnosis by metagenomic analysis of bacteria- and archaea-derived extracellular vesicles.

However, the technical goals of the present invention are not limited to the aforementioned goals, and other unmentioned technical goals will be clearly understood by those of ordinary skill in the art from the following description.

### Technical Solution

According to an aspect of the present invention, there is provided a method of providing information for breast cancer diagnosis, comprising the following processes:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria- and archaea-derived extracellular vesicles of the subject sample with that of a normal individual-derived sample through sequencing of a product of the PCR.

The present invention also provides a method of diagnosing breast cancer, comprising the following processes:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria- and archaea-derived extracellular vesicles of the subject sample with that of a normal individual-derived sample through sequencing of a product of the PCR.

The present invention also provides a method of predicting a risk for breast cancer, comprising the following processes:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria- and archaea-derived extracellular vesicles of the subject sample with that of a normal individual-derived sample through sequencing of a product of the PCR.

In one embodiment of the present invention, in process (c), breast cancer may be diagnosed by comparing, between blood samples, an increase or decrease in content of extracellular vesicles derived from one or more bacteria selected from the group consisting of the phylum Fusobacteria and the phylum Bacteroidetes.

In another embodiment of the present invention, in process (c), breast cancer may be diagnosed by comparing, between blood samples, an increase or decrease in content of extracellular vesicles derived from one or more bacteria selected from the group consisting of the class Fusobacteriia, the class Alphaproteobacteria, the class Chloroplast, the class TM7-3, and the class Bacteroidia.

In another embodiment of the present invention, in process (c), breast cancer may be diagnosed by comparing, between blood samples, an increase or decrease in content of extracellular vesicles derived from one or more bacteria selected from the group consisting of the order Fusobacteriales, the order Sphingomonadales, the order Neisseriales, the order Rhizobiales, the order Rhodospirillales, the order Actinomycetales, the order Bacillales, the order Streptophyta, the order Caulobacterales, the order Pseudomonadales, the order Bacteroidales, the order Enterobacteriales, and the order Bifidobacteriales.

In another embodiment of the present invention, in process (c), breast cancer may be diagnosed by comparing, between blood samples, an increase or decrease in content of extracellular vesicles derived from one or more bacteria selected from the group consisting of the family Staphylococcaceae, the family Fusobacteriaceae, the family Actinomycetaceae, the family Brevibacteriaceae, the family Peptostreptococcaceae, the family Rhizobiaceae, the family Corynebacteriaceae, the family Methylobacteriaceae, the family Propionibacteriaceae, the family Sphingomonadaceae, the family Neisseriaceae, the family Flavobacteriaceae, the family Intrasporangiaceae, the family Nocardiaceae, the family Caulobacteraceae, the family Micrococcaceae, the family Oxalobacteraceae, the family Moraxellaceae, the family Pseudomonadaceae, the family Porphyromonadaceae, the family Clostridiaceae, the family Rikenellaceae, the family Veillonellaceae, the family Enterobacteriaceae, the family S24-7, the family Bacteroidaceae, and the family Bifidobacteriaceae.

In another embodiment of the present invention, in process (c), breast cancer may be diagnosed by comparing, between blood samples, an increase or decrease in content of extracellular vesicles derived from one or more bacteria selected from the group consisting of the genus Hydrogenophilus, the genus Staphylococcus, the genus Fusobacterium, the genus Actinomyces, the genus Brevibacterium, the genus Granulicatella, the genus Neisseria, the genus Rothia, the genus Corynebacterium, the genus Brevundimonas, the genus Propionibacterium, the genus Porphyromonas, the genus Sphingomonas, the genus Methylobacterium, the genus Micrococcus, the genus Coprococcus, the genus Rhodococcus, the genus Cupriavidus, the genus Acinetobacter, the genus Pseudomonas, the genus Enhydrobacter, the genus Ruminococcus, the genus Dialister, the genus Tepidimonas, the genus Veillonella, the genus Phascolarctobacterium, the genus Lachnospira, the genus Klebsiella, the genus Roseburia, the genus Parabacteroides, the genus Bacteroides, the genus Bifidobacterium, the genus Megamonas, and the genus Enterobacter.

In another embodiment of the present invention, in process (c), breast cancer may be diagnosed by comparing, between urine samples, an increase or decrease in content of extracellular vesicles derived from one or more bacteria selected from the group consisting of the phylum Tenericutes, the phylum Armatimonadetes, the phylum Cyanobacteria, the phylum Verrucomicrobia, the phylum TM7, and the phylum Fusobacteria.

In another embodiment of the present invention, in process (c), breast cancer may be diagnosed by comparing, between urine samples, an increase or decrease in content of extracellular vesicles derived from one or more bacteria selected from the group consisting of the class Mollicutes, the class Chloroplast, the class Fimbriimonadia, the class TM7-3, the class Verrucomicrobiae, the class Saprospirae, the class Fusobacteriia, and the class 4C0d-2.

In another embodiment of the present invention, in process (c), breast cancer may be diagnosed by comparing, between urine samples, an increase or decrease in content of extracellular vesicles derived from one or more bacteria selected from the group consisting of the order Stramenopiles, the order RF39, the order Streptophyta, the order Fimbriimonadales, the order Verrucomicrobiales, the order Saprospirales, the order Pseudomonadales, the order Fusobacteriales, the order Burkholderiales, the order Bifidobacteriales, the order Oceanospirillales, and the order YS2.

In another embodiment of the present invention, in process (c), breast cancer may be diagnosed by comparing, between urine samples, an increase or decrease in content of extracellular vesicles derived from one or more bacteria selected from the group consisting of the family Exiguobacteraceae, the family Cellulomonadaceae, the family F16, the family Fimbriimonadaceae, the family Oxalobacteraceae, the family Streptomycetaceae, the family Carnobacteriaceae, the family Actinomycetaceae, the family Nocardiaceae, the family Peptococcaceae, the family Fusobacteriaceae, the family Mogibacteriaceae, the family Pseudomonadaceae, the family Verrucomicrobiaceae, the family Bradyrhizobiaceae, the family Enterococcaceae, the family Chitinophagaceae, the family Moraxellaceae, the family Rhizobiaceae, the family Ruminococcaceae, the family Bacteroidaceae, the family Dermacoccaceae, the family Gordoniaceae, the family Acetobacteraceae, the family Bifidobacteriaceae, the family Comamonadaceae, the family Barnesiellaceae, the family Peptostreptococcaceae, the family Halomonadaceae, the family Rikenellaceae, and the family Methylocystaceae.

In another embodiment of the present invention, in process (c), breast cancer may be diagnosed by comparing, between urine samples, an increase or decrease in content of extracellular vesicles derived from one or more bacteria selected from the group consisting of the genus Ralstonia, the genus Rhizobium, the genus Morganella, the genus Tetragenococcus, the genus Exiguobacterium, the genus Streptomyces, the genus Oribacterium, the genus Sporosarcina, the genus Jeotgalicoccus, the genus Fimbriimonas, the genus Enterococcus, the genus Porphyromonas, the genus Lactococcus, the genus Cellulomonas, the genus Proteus, the genus Granulicatella, the genus Acinetobacter, the genus Actinomyces, the genus Cupriavidus, the genus Rhodococcus, the genus Fusobacterium, the genus Pseudomonas, the genus Akkermansia, the genus Leptotrichia, the genus Methylobacterium, the genus Weissella, the genus Bacteroides, the genus Veillonella, the genus Dermacoccus, the genus Coprococcus, the genus Ruminococcus, the genus Trabulsiella, the genus Gordonia, the genus Lachnospira, the genus Klebsiella, the genus Enterobacter, the genus Faecalibacterium, the genus Serratia, the genus Bifidobacterium, the genus Citrobacter, the genus Bilophila, the genus Virgibacillus, the genus Halomonas, the genus Roseburia, the genus Comamonas, the genus Methylopila, and the genus Gemella.

In another embodiment of the present invention, the subj ect sample may be blood or urine.

In another embodiment of the present invention, the blood may be whole blood, serum, plasma, or blood mononuclear cells.

### Advantageous Effects

Extracellular vesicles secreted from bacteria and archaea present in the environment are absorbed into the human body, and thus may directly affect the occurrence of cancer, and it is difficult to diagnose breast cancer early before symptoms occur, and thus efficient treatment therefor is difficult. Thus, according to the present invention, a risk of developing breast cancer can be predicted through metagenomic analysis of bacteria-derived extracellular vesicles by using a human body-derived sample, and thus the onset of breast cancer can be delayed or breast cancer can be prevented through appropriate management by early diagnosis and prediction of a risk group for breast cancer, and, even after breast cancer occurs, early diagnosis for breast cancer can be implemented, thereby lowering the incidence rate of breast cancer and increasing therapeutic effects. In addition, patients diagnosed with breast cancer are able to avoid exposure to causative factors predicted by metagenomic analysis, whereby the progression of cancer can be ameliorated, or recurrence of breast cancer can be prevented.

### DESCRIPTION OF DRAWINGS

FIG. 1A illustrates images showing the distribution pattern of bacteria and extracellular vesicles over time after intestinal bacteria and bacteria-derived extracellular vesicles (EVs) were orally administered to mice, and FIG. 1B illustrates images showing the distribution pattern of bacteria and EVs after being orally administered to mice and, at 12 hours, blood and various organs were extracted.

FIG. 2 illustrates distribution results of bacteria-derived EVs exhibiting significant diagnostic performance at a phylum level, after metagenomic analysis of bacteria-derived EVs isolated from breast cancer patient-derived blood and normal individual-derived blood.

FIG. 3 illustrates distribution results of bacteria-derived EVs exhibiting significant diagnostic performance at a class level, after metagenomic analysis of bacteria-derived EVs isolated from breast cancer patient-derived blood and normal individual-derived blood.

FIG. 4 illustrates distribution results of bacteria-derived EVs exhibiting significant diagnostic performance at an order level, after metagenomic analysis of bacteria-derived EVs isolated from breast cancer patient-derived blood and normal individual-derived blood.

FIG. 5 illustrates distribution results of bacteria-derived EVs exhibiting significant diagnostic performance at a family level, after metagenomic analysis of bacteria-derived EVs isolated from breast cancer patient-derived blood and normal individual-derived blood.

FIG. 6 illustrates distribution results of bacteria-derived EVs exhibiting significant diagnostic performance at a genus level, after metagenomic analysis of bacteria-derived EVs isolated from breast cancer patient-derived blood and normal individual-derived blood.

FIG. 7 illustrates distribution results of bacteria-derived EVs exhibiting significant diagnostic performance at a phylum level, after metagenomic analysis of bacteria-derived EVs isolated from breast cancer patient-derived urine and normal individual-derived urine.

FIG. 8 illustrates distribution results of bacteria-derived EVs exhibiting significant diagnostic performance at a class level, after metagenomic analysis of bacteria-derived EVs isolated from breast cancer patient-derived urine and normal individual-derived urine.

FIG. 9 illustrates distribution results of bacteria-derived EVs exhibiting significant diagnostic performance at an order level, after metagenomic analysis of bacteria-derived EVs isolated from breast cancer patient-derived urine and normal individual-derived urine.

FIG. 10 illustrates distribution results of bacteria-derived EVs exhibiting significant diagnostic performance at a family level, after metagenomic analysis of bacteria-derived EVs isolated from breast cancer patient-derived urine and normal individual-derived urine.

FIG. 11 illustrates distribution results of bacteria-derived EVs exhibiting significant diagnostic performance at a genus level, after metagenomic analysis of bacteria-derived EVs isolated from breast cancer patient-derived urine and normal individual-derived urine.

### BEST MODEL

The present invention relates to a method of diagnosing breast cancer through bacterial and archaeal metagenomic analysis. The inventors of the present invention extracted genes from bacteria- and archaea-derived extracellular vesicles using a subject-derived sample, performed metagenomic analysis thereon, and identified bacteria-derived extracellular vesicles capable of acting as a causative factor of breast cancer.

Thus, the present invention provides a method of providing information for breast cancer diagnosis, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria- and archaea-derived extracellular vesicles of the subject sample with that of a normal individual-derived sample through sequencing of a product of the PCR.

The term “breast cancer diagnosis” as used herein refers to determining whether a patient has a risk for breast cancer, whether the risk for breast cancer is relatively high, or whether breast cancer has already occurred. The method of the present invention may be used to delay the onset of breast cancer through special and appropriate care for a specific patient, which is a patient having a high risk for breast cancer or prevent the onset of breast cancer. In addition, the method may be clinically used to determine treatment by selecting the most appropriate treatment method through early diagnosis of breast cancer.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, blood and urine.

In the present invention, the subject sample may be blood or urine, and the blood may be whole blood, serum, plasma, or blood mononuclear cells, but the present invention is not limited thereto.

In an embodiment of the present invention, metagenomic analysis is performed on the bacteria- and archaea-derived extracellular vesicles, and bacteria-derived extracellular vesicles capable of acting as a cause of the onset of breast cancer were actually identified by analysis at phylum, class, order, family, and genus levels.

More particularly, in one embodiment of the present invention, as a result of performing bacterial metagenomic analysis on extracellular vesicles present in subject-derived blood samples at a phylum level, the content of extracellular vesicles derived from bacteria belonging to the phylum Fusobacteria and the phylum Bacteroidetes was significantly different between breast cancer patients and normal individuals (see Example 4).

More particularly, in one embodiment of the present invention, as a result of performing bacterial metagenomic analysis on extracellular vesicles present in subject-derived blood samples at a class level, the content of extracellular vesicles derived from bacteria belonging to the class Fusobacteriia, the class Alphaproteobacteria, the class Chloroplast, the class TM7-3, and the class Bacteroidia was significantly different between breast cancer patients and normal individuals (see Example 4).

More particularly, in one embodiment of the present invention, as a result of performing bacterial metagenomic analysis on extracellular vesicles present in subject-derived blood samples at an order level, the content of extracellular vesicles derived from bacteria belonging to the order Fusobacteriales, the order Sphingomonadales, the order Neisseriales, the order Rhizobiales, the order Rhodospirillales, the order Actinomycetales, the order Bacillales, the order Streptophyta, the order Caulobacterales, the order Pseudomonadales, the order Bacteroidales, the order Enterobacteriales, and the order Bifidobacteriales was significantly different between breast cancer patients and normal individuals (see Example 4).

More particularly, in one embodiment of the present invention, as a result of performing bacterial metagenomic analysis on extracellular vesicles present in subject-derived blood samples at a family level, the content of extracellular vesicles derived from bacteria belonging to the family Staphylococcaceae, the family Fusobacteriaceae, the family Actinomycetaceae, the family Brevibacteriaceae, the family Peptostreptococcaceae, the family Rhizobiaceae, the family Corynebacteriaceae, the family Methylobacteriaceae, the family Propionibacteriaceae, the family Sphingomonadaceae, the family Neisseriaceae, the family Flavobacteriaceae, the family Intrasporangiaceae, the family Nocardiaceae, the family Caulobacteraceae, the family Micrococcaceae, the family Oxalobacteraceae, the family Moraxellaceae, the family Pseudomonadaceae, the family Porphyromonadaceae, the family Clostridiaceae, the family Rikenellaceae, the family Veillonellaceae, the family Enterobacteriaceae, the family S24-7, the family Bacteroidaceae, and the family Bifidobacteriaceae was significantly different between breast cancer patients and normal individuals (see Example 4).

More particularly, in one embodiment of the present invention, as a result of performing bacterial metagenomic analysis on extracellular vesicles present in subject-derived blood samples at a genus level, the content of extracellular vesicles derived from bacteria belonging to the genus Hydrogenophilus, the genus Staphylococcus, the genus Fusobacterium, the genus Actinomyces, the genus Brevibacterium, the genus Granulicatella, the genus Neisseria, the genus Rothia, the genus Corynebacterium, the genus Brevundimonas, the genus Propionibacterium, the genus Porphyromonas, the genus Sphingomonas, the genus Methylobacterium, the genus Micrococcus, the genus Coprococcus, the genus Rhodococcus, the genus Cupriavidus, the genus Acinetobacter, the genus Pseudomonas, the genus Enhydrobacter, the genus Ruminococcus, the genus Dialister, the genus Tepidimonas, the genus Veillonella, the genus Phascolarctobacterium, the genus Lachnospira, the genus Klebsiella, the genus Roseburia, the genus Parabacteroides, the genus Bacteroides, the genus Bifidobacterium, the genus Megamonas, and the genus Enterobacter was significantly different between breast cancer patients and normal individuals (see Example 4).

More particularly, in one embodiment of the present invention, as a result of performing bacterial metagenomic analysis on extracellular vesicles present in subject-derived urine samples at a phylum level, the content of extracellular vesicles derived from bacteria belonging to the phylum Tenericutes, the phylum Armatimonadetes, the phylum Cyanobacteria, the phylum Verrucomicrobia, the phylum TM7, and the phylum Fusobacteria was significantly different between breast cancer patients and normal individuals (see Example 5).

More particularly, in one embodiment of the present invention, as a result of performing bacterial metagenomic analysis on extracellular vesicles present in subject-derived urine samples at a class level, the content of extracellular vesicles derived from bacteria belonging to the class Mollicutes, the class Chloroplast, the class Fimbriimonadia, the class TM7-3, the class Verrucomicrobiae, the class Saprospirae, the class Fusobacteriia, and the class 4C0d-2 was significantly different between breast cancer patients and normal individuals (see Example 5).

More particularly, in one embodiment of the present invention, as a result of performing bacterial metagenomic analysis on extracellular vesicles present in subject-derived urine samples at an order level, the content of extracellular vesicles derived from bacteria belonging to the order Stramenopiles, the order RF39, the order Streptophyta, the order Fimbriimonadales, the order Verrucomicrobiales, the order Saprospirales, the order Pseudomonadales, the order Fusobacteriales, the order Burkholderiales, the order Bifidobacteriales, the order Oceanospirillales, and the order YS2 was significantly different between breast cancer patients and normal individuals (see Example 5).

More particularly, in one embodiment of the present invention, as a result of performing bacterial metagenomic analysis on extracellular vesicles present in subject-derived urine samples at a family level, the content of extracellular vesicles derived from bacteria belonging to the family Exiguobacteraceae, the family Cellulomonadaceae, the family F16, the family Fimbriimonadaceae, the family Oxalobacteraceae, the family Streptomycetaceae, the family Carnobacteriaceae, the family Actinomycetaceae, the family Nocardiaceae, the family Peptococcaceae, the family Fusobacteriaceae, the family Mogibacteriaceae, the family Pseudomonadaceae, the family Verrucomicrobiaceae, the family Bradyrhizobiaceae, the family Enterococcaceae, the family Chitinophagaceae, the family Moraxellaceae, the family Rhizobiaceae, the family Ruminococcaceae, the family Bacteroidaceae, the family Dermacoccaceae, the family Gordoniaceae, the family Acetobacteraceae, the family Bifidobacteriaceae, the family Comamonadaceae, the family Barnesiellaceae, the family Peptostreptococcaceae, the family Halomonadaceae, the family Rikenellaceae, and the family Methylocystaceae was significantly different between breast cancer patients and normal individuals (see Example 5).

More particularly, in one embodiment of the present invention, as a result of performing bacterial metagenomic analysis on extracellular vesicles present in subject-derived urine samples at a genus level, the content of extracellular vesicles derived from bacteria belonging to the genus Ralstonia, the genus Rhizobium, the genus Morganella, the genus Tetragenococcus, the genus Exiguobacterium, the genus Streptomyces, the genus Oribacterium, the genus Sporosarcina, the genus Jeotgalicoccus, the genus Fimbriimonas, the genus Enterococcus, the genus Porphyromonas, the genus Lactococcus, the genus Cellulomonas, the genus Proteus, the genus Granulicatella, the genus Acinetobacter, the genus Actinomyces, the genus Cupriavidus, the genus Rhodococcus, the genus Fusobacterium, the genus Pseudomonas, the genus Akkermansia, the genus Leptotrichia, the genus Methylobacterium, the genus Weissella, the genus Bacteroides, the genus Veillonella, the genus Dermacoccus, the genus Coprococcus, the genus Ruminococcus, the genus Trabulsiella, the genus Gordonia, the genus Lachnospira, the genus Klebsiella, the genus Enterobacter, the genus Faecalibacterium, the genus Serratia, the genus Bifidobacterium, the genus Citrobacter, the genus Bilophila, the genus Virgibacillus, the genus Halomonas, the genus Roseburia, the genus Comamonas, the genus Methylopila, and the genus Gemella was significantly different between breast cancer patients and normal individuals (see Example 5).

From the above-described example results, it can be confirmed that bacteria-derived extracellular vesicles exhibiting a significant change in content in breast cancer patients compared to normal individuals, are identified by performing bacterial metagenomic analysis on bacteria-derived extracellular vesicles isolated from blood and urine, and breast cancer may be diagnosed by analyzing an increase or decrease in the content of bacteria-derived extracellular vesicles at each level through metagenomic analysis.

Hereinafter, the present invention will be described with reference to exemplary examples to aid in understanding of the present invention. However, these examples are provided only for illustrative purposes and are not intended to limit the scope of the present invention.

### EXAMPLES

### Example 1. Analysis of In Vivo Absorption, Distribution, and Excretion Patterns of Intestinal Bacteria and Bacteria-Derived Extracellular Vesicles

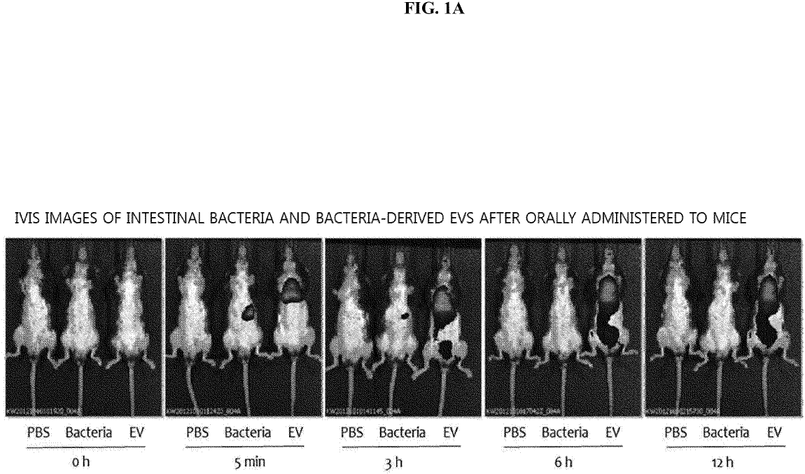

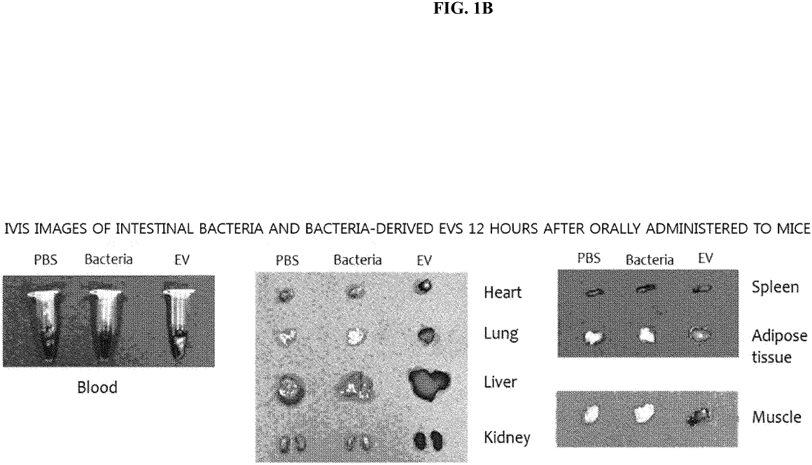

To evaluate whether intestinal bacteria and bacteria-derived extracellular vesicles are systematically absorbed through the gastrointestinal tract, an experiment was conducted using the following method. More particularly, 50 µg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 µg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2. Vesicle Isolation and DNA Extraction From Blood and Urine

To isolate extracellular vesicles and extract DNA, from blood and urine, first, blood or urine was added to a 10 ml tube and centrifuged at 3,500 x g and 4° C. for 10 min to precipitate a suspension, and only a supernatant was collected, which was then placed in a new 10 ml tube. The collected supernatant was filtered using a 0.22 µm filter to remove bacteria and impurities, and then placed in centripreigugal filters (50 kD) and centrifuged at 1500 x g and 4° C. for 15 min to discard materials with a smaller size than 50 kD, and then concentrated to 10 ml. Once again, bacteria and impurities were removed therefrom using a 0.22 µm filter, and then the resulting concentrate was subjected to ultra-high speed centrifugation at 150,000 x g and 4° C. for 3 hours by using a Type 90ti rotor to remove a supernatant, and the agglomerated pellet was dissolved with phosphate-buffered saline (PBS), thereby obtaining vesicles.

100 µl of the extracellular vesicles isolated from the blood and urine according to the above-described method was boiled at 100° C. to allow the internal DNA to come out of the lipid and then cooled on ice. Next, the resulting vesicles were centrifuged at 10,000 x g and 4° C. for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16 s rDNA primers shown in Table 1 below.

TABLE 1

| Primer | | Sequence | SEQ ID NO. |
|---|---|---|---|
| 16S rDNA | 16S_V3_F | 5′-TCGTCGGCAGCGTCAGATGTGTATAAGAGACAGCCTACGGGNGGCWGCAG-3′ | 1 |
| | 16S_V4_R | 5′-GTCTCGTGGGCTCGGAGATGTGTATAAGAGACAGGACTACHVGG | 2 |

### Example 3. Metagenomic Analysis Using DNA Extracted From Blood and Urine

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score <20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4. Breast Cancer Diagnostic Model Based on Metagenomic Analysis of Bacteria-Derived EVs Isolated from Blood

EVs were isolated from blood samples of 96 breast cancer patients and 192 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

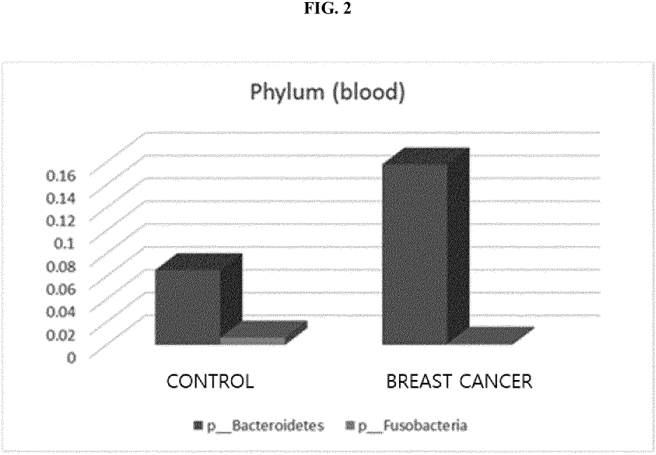

As a result of analyzing bacteria-derived EVs in blood at a phylum level, a diagnostic model developed using bacteria belonging to the phylum Fusobacteria and the phylum Bacteroidetes as a biomarker exhibited significant diagnostic performance for breast cancer (see Table 2 and FIG. 2).

TABLE 2

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| P_Fusobacteria | 0.0063 | 0.0101 | 0.0010 | 0.0025 | 0.0000 | 0.16 | 0.70 | 0.99 | 0.02 | 0.74 | 0.98 | 0.06 |
| p_Bacteroidetes | 0.0654 | 0.0352 | 0.1578 | 0.0419 | 0.0000 | 2.41 | 0.95 | 0.94 | 0.76 | 0.98 | 0.96 | 0.82 |

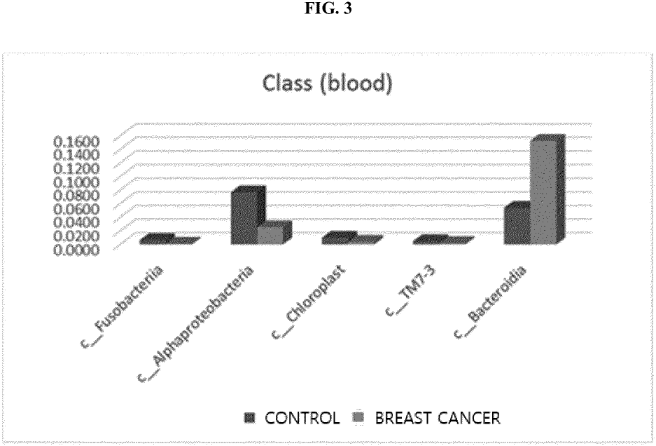

As a result of analyzing bacteria-derived EVs in blood at a class level, a diagnostic model developed using bacteria belonging to the class Fusobacteriia, the class Alphaproteobacteria, the class Chloroplast, the class TM7-3, and the class Bacteroidia as a biomarker exhibited significant diagnostic performance for breast cancer (see Table 3 and FIG. 3).

TABLE 3

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| c_Fusobacteriia | 0.0063 | 0.0101 | 0.0010 | 0.0025 | 0.0000 | 0.16 | 0.71 | 0.94 | 0.10 | 0.71 | 0.94 | 0.09 |
| c_Alphaproteobacteria | 0.0768 | 0.0480 | 0.0251 | 0.0230 | 0.0000 | 0.33 | 0.88 | 0.88 | 0.69 | 0.83 | 0.87 | 0.62 |
| c_Chloroplast | 0.0098 | 0.0141 | 0.0038 | 0.0046 | 0.0000 | 0.38 | 0.70 | 0.96 | 0.13 | 0.58 | 0.89 | 0.03 |
| c_TM7-3 | 0.0046 | 0.0076 | 0.0020 | 0.0044 | 0.0002 | 0.43 | 0.67 | 0.99 | 0.02 | 0.53 | 0.98 | 0.00 |
| c_Bacteroidia | 0.0538 | 0.0366 | 0.1525 | 0.0441 | 0.0000 | 2.84 | 0.96 | 0.93 | 0.76 | 0.95 | 0.98 | 0.82 |

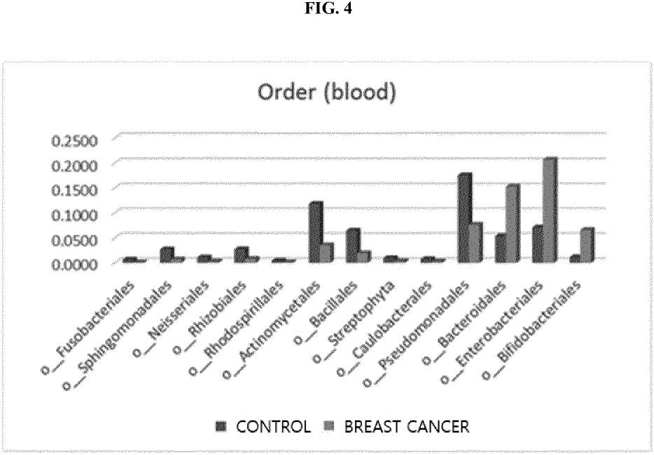

As a result of analyzing bacteria-derived EVs in blood at an order level, a diagnostic model developed using bacteria belonging to the order Fusobacteriales, the order Sphingomonadales, the order Neisseriales, the order Rhizobiales, the order Rhodospirillales, the order Actinomycetales, the order Bacillales, the order Streptophyta, the order Caulobacterales, the order Pseudomonadales, the order Bacteroidales, the order Enterobacteriales, and the order Bifidobacteriales as a biomarker exhibited significant diagnostic performance for breast cancer (see Table 4 and FIG. 4).

TABLE 4

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| o_Fusobacteriales | 0.0063 | 0.0101 | 0.0010 | 0.0025 | 0.0000 | 0.16 | 0.71 | 0.94 | 0.10 | 0.71 | 0.94 | 0.09 |
| o_Sphingomonadales | 0.0267 | 0.0268 | 0.0069 | 0.0093 | 0.0000 | 0.26 | 0.85 | 0.83 | 0.66 | 0.78 | 0.87 | 0.44 |
| o_Neisseriales | 0.0107 | 0.0186 | 0.0030 | 0.0055 | 0.0000 | 0.28 | 0.69 | 0.99 | 0.05 | 0.56 | 0.98 | 0.03 |
| o_Rhizobiales | 0.0270 | 0.0223 | 0.0079 | 0.0096 | 0.0000 | 0.29 | 0.86 | 0.87 | 0.73 | 0.76 | 0.77 | 0.68 |
| o_Rhodospirillales | 0.0042 | 0.0085 | 0.0012 | 0.0025 | 0.0000 | 0.29 | 0.68 | 0.99 | 0.03 | 0.62 | 1.00 | 0.03 |
| o_Aetinomycetules | 0.1178 | 0.0731 | 0.0351 | 0.0128 | 0.0000 | 0.30 | 0.85 | 0.84 | 0.63 | 0.84 | 0.81 | 0.68 |
| o_Bacillales | 0.0644 | 0.0559 | 0.0193 | 0.0189 | 0.0000 | 0.30 | 0.84 | 0.82 | 0.58 | 0.79 | 0.81 | 0.62 |
| o_Streptophyta | 0.0095 | 0.0139 | 0.0035 | 0.0046 | 0.0000 | 0.37 | 0.71 | 0.96 | 0.15 | 0.59 | 0.89 | 0.03 |
| o_Caulobaclerales | 0.0074 | 0.0111 | 0.0029 | 0.0029 | 0.0000 | 0.39 | 0.69 | 1.00 | 0.03 | 0.58 | 0.96 | 0.003 |
| o_Pseudomonadales | 0.1752 | 0.1017 | 0.0763 | 0.0502 | 0.0000 | 0.44 | 0.84 | 0.83 | 0.60 | 0.82 | 0.81 | 0.47 |
| o_Bacteroidales | 0.0538 | 0.0366 | 0.1525 | 0.0441 | 0.0000 | 2.84 | 0.96 | 0.93 | 0.76 | 0.95 | 0.98 | 0.82 |
| o_Enterobacteriales | 0.0708 | 0.0476 | 0.2066 | 0.0917 | 0.0000 | 2.92 | 0.87 | 0.94 | 0.66 | 0.93 | 0.91 | 0.62 |
| o_Bifidobacterials | 0.9115 | 0.0128 | 0.0659 | 0.0320 | 0.0000 | 5.75 | 0.94 | 0.98 | 0.79 | 0.94 | 0.98 | 0.82 |

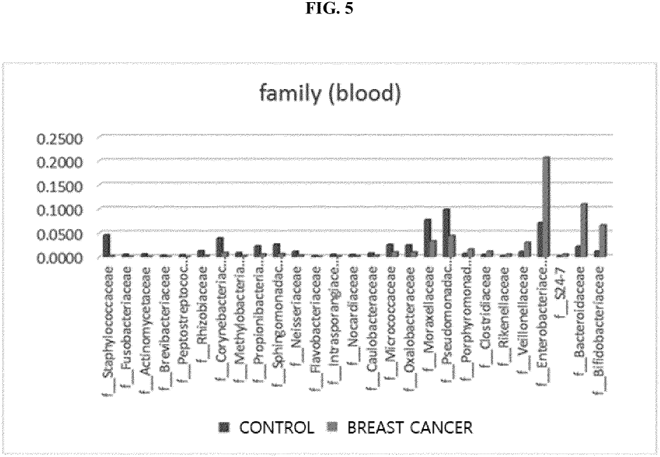

As a result of analyzing bacteria-derived EVs in blood at a family level, a diagnostic model developed using bacteria belonging to the family Staphylococcaceae, the family Fusobacteriaceae, the family Actinomycetaceae, the family Brevibacteriaceae, the family Peptostreptococcaceae, the family Rhizobiaceae, the family Corynebacteriaceae, the family Methylobacteriaceae, the family Propionibacteriaceae, the family Sphingomonadaceae, the family Neisseriaceae, the family Flavobacteriaceae, the family Intrasporangiaceae, the family Nocardiaceae, the family Caulobacteraceae, the family Micrococcaceae, the family Oxalobacteraceae, the family Moraxellaceae, the family Pseudomonadaceae, the family Porphyromonadaceae, the family Clostridiaceae, the family Rikenellaceae, the family Veillonellaceae, the family Enterobacteriaceae, the family S24-7, the family Bacteroidaceae, and the family Bifidobacteriaceae as a biomarker exhibited significant diagnostic performance for breast cancer (see Table 5 and FIG. 5).

TABLE 5

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| f_Staphylococcaccae | 0.0451 | 0.0491 | 0.0019 | 0.0031 | 0.0000 | 0.04 | 0.92 | 0.85 | 0.92 | 0.96 | 0.89 | 0.9191 |
| f_Fusobacteriaceae | 0.0048 | 0.0085 | 0.0006 | 0.0014 | 0.0000 | 0.12 | 0.69 | 0.96 | 0.10 | 0.74 | 0.96 | 0.09 |
| f_Actinomycetaceae | 0.0054 | 0.0087 | 0.0007 | 0.0010 | 0.0000 | 0.13 | 0.72 | 0.78 | 0.35 | 0.75 | 0.77 | 0.47 |
| f_Brevibacteriaceae | 0.0028 | 0.0054 | 0.0004 | 0.0008 | 0.0000 | 0.13 | 0.72 | 0.99 | 0.08 | 0.65 | 1.00 | 0.00 |
| f_Peptostreptococeaceae | 0.0037 | 0.0106 | 0.0006 | 0.0018 | 0.0001 | 0.16 | 0.72 | 0.98 | 0.15 | 0.63 | 0.98 | 0.09 |
| f_Rhizobiaceae | 0.0123 | 0.0160 | 0.0026 | 0.0052 | 0.0000 | 0.21 | 0.84 | 0.80 | 0.73 | 0.73 | 0.72 | 0.71 |
| f_Corynebacteriaceae | 0.0389 | 0.0338 | 0.0086 | 0.0088 | 0.0000 | 0.22 | 0.78 | 0.79 | 0.53 | 0.85 | 0.83 | 0.68 |
| f_Methylobacteriaceae | 0.0082 | 0.0112 | 0.0018 | 0.0039 | 0.0000 | 0.22 | 0.76 | 0.86 | 0.39 | 0.66 | 0.77 | 0.26 |
| f_Propionibacteriaceae | 0.0224 | 0.0223 | 0.0052 | 0.0066 | 0.0000 | 0.23 | 0.79 | 0.83 | 0.48 | 0.77 | 0.81 | 0.44 |
| f_Sphingomonadaceae | 0.0258 | 0.0264 | 0.0065 | 0.0093 | 0.0000 | 0.25 | 0.85 | 0.83 | 0.66 | 0.78 | 0.87 | 0.44 |
| f_Neisseriaceae | 0.0107 | 0.0187 | 0.0030 | 0.0055 | 0.0000 | 0.28 | 0.69 | 0.99 | 0.05 | 0.56 | 0.98 | 0.03 |
| f_Flavobacteriaceae | 0.0018 | 0.0034 | 0.0006 | 0.0017 | 0.0000 | 0.31 | 0.64 | 0.99 | 0.02 | 0.51 | 1.00 | 0.00 |
| f_Intrasporangiaceae | 0.0047 | 0.0074 | 0.0017 | 0.0029 | 0.0000 | 0.36 | 0.65 | 0.99 | 0.00 | 0.59 | 1.00 | 0.00 |
| f_Nocardiaceae | 0.0045 | 0.0089 | 0.0016 | 0.0030 | 0.0001 | 0.36 | 0.63 | 0.99 | 0.00 | 0.53 | 0.98 | 0.00 |
| f_Caulobacteraceae | 0.0074 | 0.0111 | 0.0029 | 0.0062 | 0.0000 | 0.39 | 0.69 | 1.00 | 0.03 | 0.58 | 0.96 | 0.03 |
| f_Micrococcaceae | 0.0251 | 0.0243 | 0.0099 | 0.0121 | 0.0000 | 0.39 | 0.76 | 0.86 | 0.27 | 0.72 | 0.87 | 0.38 |
| f_Oxalobacteraceae | 0.0243 | 0.0388 | 0.0099 | 0.0089 | 0.0000 | 0.41 | 0.69 | 0.95 | 0.18 | 0.54 | 0.91 | 0.03 |
| f_Moraxellaceae | 0.0768 | 0.0547 | 0.0326 | 0.0273 | 0.0000 | 0.43 | 0.83 | 0.83 | 0.53 | 0.76 | 0.79 | 0.56 |
| f_Pseudomonadaceae | 0.0984 | 0.0812 | 0.0437 | 0.0363 | 0.0000 | 0.44 | 0.80 | 0.90 | 0.50 | 0.76 | 0.89 | 0.47 |
| f_Porphymmonadaceae | 0.0066 | 0.0087 | 0.0157 | 0.0137 | 0.0000 | 2.37 | 0.78 | 0.94 | 0.19 | 0.82 | 0.96 | 0.18 |
| f_Clostridiaceae | 0.0045 | 0.0061 | 0.0115 | 0.0141 | 0.0000 | 2.52 | 0.73 | 0.96 | 0.27 | 0.56 | 0.98 | 0.18 |
| f_Rikenellaceae | 0.0019 | 0.0042 | 0.0052 | 0.0086 | 0.0007 | 2.67 | 0.70 | 0.97 | 0.16 | 0.52 | 0.96 | 0.03 |
| f_Veillonell aceae | 0.0106 | 0.0107 | 0.0296 | 0.0377 | 0.0000 | 2.80 | 0.83 | 0.94 | 0.52 | 0.70 | 0.87 | 0.44 |
| f_Enterobacteriaceae | 0.0708 | 0.0476 | 0.2066 | 0.0917 | 0.0000 | 2.92 | 0.87 | 0.94 | 0.66 | 0.93 | 0.91 | 0.62 |
| f_S24-7 | 0.0014 | 0.0037 | 0.0054 | 0.0125 | 0.0033 | 3.82 | 0.63 | 0.96 | 0.11 | 0.56 | 1.00 | 0.12 |
| f_Bacteroidaceae | 0.0217 | 0.0229 | 0.1098 | 0.0441 | 0.0000 | 5.07 | 0.98 | 0.96 | 0.90 | 0.95 | 0.94 | 0.79 |
| f_Bifidobacteriaceae | 0.0115 | 0.0128 | 0.0659 | 0.0320 | 0.0000 | 5.75 | 0.94 | 0.98 | 0.79 | 0.94 | 0.98 | 0.82 |

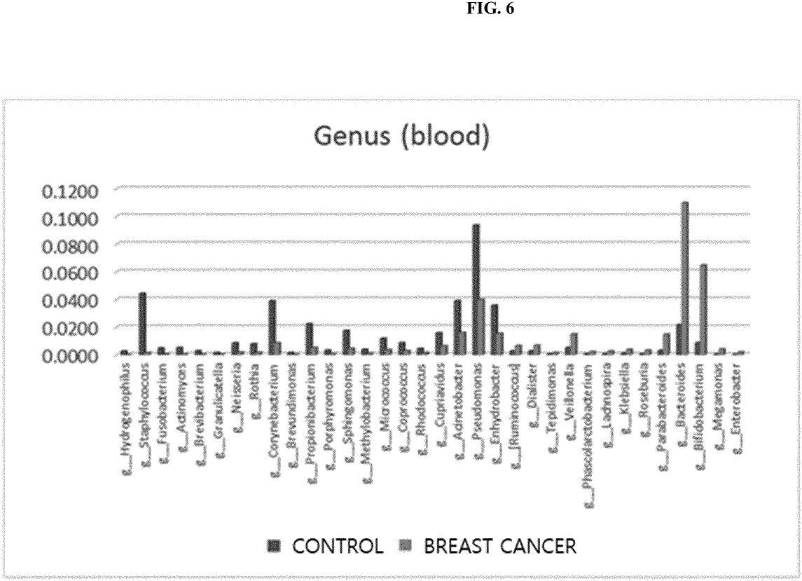

As a result of analyzing bacteria-derived EVs in blood at a genus level, a diagnostic model developed using bacteria belonging to the genus Hydrogenophilus, the genus Staphylococcus, the genus Fusobacterium, the genus Actinomyces, the genus Brevibacterium, the genus Granulicatella, the genus Neisseria, the genus Rothia, the genus Corynebacterium, the genus Brevundimonas, the genus Propionibacterium, the genus Porphyromonas, the genus Sphingomonas, the genus Methylobacterium, the genus Micrococcus, the genus Coprococcus, the genus Rhodococcus, the genus Cupriavidus, the genus Acinetobacter, the genus Pseudomonas, the genus Enhydrobacter, the genus Ruminococcus, the genus Dialister, the genus Tepidimonas, the genus Veillonella, the genus Phascolarctobacterium, the genus Lachnospira, the genus Klebsiella, the genus Roseburia, the genus Parabacteroides, the genus Bacteroides, the genus Bifidobacterium, the genus Megamonas, and the genus Enterobacter as a biomarker exhibited significant diagnostic performance for breast cancer (see Table 6 and FIG. 6).

TABLE 6

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| g_Hydroge nophilus | 0.0026 | 0.0077 | 0.0000 | 0.0001 | 0.0000 | 0.00 | 0.74 | 0.99 | 0.06 | 0.69 | 0.98 | 0.03 |
| g_Staphylococcus | 0.0443 | 0.0489 | 0.0015 | 0.0024 | 0.0000 | 0.03 | 0.93 | 0.86 | 0.92 | 0.96 | 0.92 | 0.91 |
| g_Fusobacterium | 0.0048 | 0.0085 | 0.0006 | 0.0014 | 0.0000 | 0.12 | 0.69 | 0.96 | 0.10 | 0.74 | 0.96 | 0.09 |
| g_Actinomyees | 0.0051 | 0.0085 | 0.0006 | 0.0009 | 0.0000 | 0.12 | 0.71 | 0.82 | 0.24 | 0.74 | 0.81 | 0.32 |
| g_Brevibacterium | 0.0028 | 0.0054 | 0.0004 | 0.0008 | 0.0000 | 0.13 | 0.72 | 0.99 | 0.08 | 0.65 | 1.00 | 0.00 |
| g_Granulieatella | 0.0016 | 0.0032 | 0.0003 | 0.0010 | 0.0000 | 0.20 | 0.69 | 0.99 | 0.05 | 0.62 | 0.98 | 0.03 |
| g_Neisseria | 0.0085 | 0.0155 | 0.0018 | 0.0045 | 0.0000 | 0.21 | 0.72 | 0.99 | 0.10 | 0.62 | 0.98 | 0.06 |
| g_Rothia | 0.008 | 0.0137 | 0.0016 | 0.0023 | 0.0000 | 0.21 | 0.73 | 0.88 | 0.24 | 0.65 | 0.85 | 0.18 |
| g_Corynebacterium | 0.0389 | 0.0338 | 0.0086 | 0.0088 | 0.0000 | 0.22 | 0.78 | 0.79 | 0.53 | 0.85 | 0.83 | 0.68 |
| g_Brevundimonas | 0.0015 | 0.0049 | 0.0003 | 0.0010 | 0.0014 | 0.22 | 0.67 | 0.99 | 0.03 | 0.50 | 0.98 | 0.03 |
| g_Propionibacterium | 0.0223 | 0.0223 | 0.0052 | 0.0066 | 0.0000 | 0.23 | 0.79 | 0.82 | 0.48 | 0.77 | 0.81 | 0.44 |
| g_Porphyromonas | 0.0032 | 0.0068 | 0.0008 | 0.0036 | 0.0001 | 0.25 | 0.69 | 0.99 | 0.02 | 0.52 | 0.98 | 0.00 |
| g_Sphingomonas | 0.0174 | 0.0213 | 0.0046 | 0.0089 | 0.0000 | 0.26 | 0.82 | 0.85 | 0.60 | 0.73 | 0.87 | 0.38 |
| g_Methylo | 0.00 | 0.00 | 0.00 | 0.00 | 0.00 | 0.2 | 0.68 | 1.00 | 0.02 | 0.5 | 1.00 | 0.0 |
| bacterium | 38.00 | 86.00 | 10.00 | 33.00 | 01.00 | 6.00 | | | | 2.00 | | 0.00 |
| g_Micrococcus | 0.0118 | 0.0164 | 0.0037 | 0.0061 | 0.0000 | 0.31 | 0.71 | 0.92 | 0.16 | 0.69 | 0.89 | 0.15 |
| g_Coprococcus | 0.0086 | 0.0110 | 0.0028 | 0.0050 | 0.0000 | 0.32 | 0.70 | 0.95 | 0.16 | 0.67 | 0.94 | 0.06 |
| g_Rhodococcus | 0.0045 | 0.0089 | 0.0016 | 0.0030 | 0.0001 | 0.36 | 0.63 | 0.99 | 0.00 | 0.53 | 0.98 | 0.00 |
| g_Cupriavidus | 0.0158 | 0.0347 | 0.0065 | 0.0059 | 0.0004 | 0.41 | 0.59 | 1.00 | 0.02 | 0.47 | 0.98 | 0.00 |
| g_Acinetohacter | 0.0390 | 0.0328 | 0.0160 | 0.0228 | 0.0000 | 0.41 | 0.80 | 0.94 | 0.44 | 0.79 | 0.89 | 0.24 |
| g_Pseudomonas | 0.0936 | 0.0811 | 0.0400 | 0.0336 | 0.0000 | 0.43 | 0.81 | 0.91 | 0.53 | 0.76 | 0.89 | 0.47 |
| g_Enhydrohacter | 0.0357 | 0.0445 | 0.0154 | 0.0122 | 0.0000 | 0.43 | 0.75 | 0.83 | 0.32 | 0.55 | 0.68 | 0.24 |
| g_[Ruminococcus] | 0.0027 | 0.0043 | 0.0066 | 0.0121 | 0.0030 | 2.45 | 0.66 | 0.96 | 0.19 | 0.50 | 0.85 | 0.12 |
| g_Dialister | 0.0027 | 0.0065 | 0.0068 | 0.0089 | 0.0001 | 2.51 | 0.73 | 0.94 | 0.15 | 0.60 | 0.94 | 0.12 |
| g_Tepidimonas | 0.0006 | 0.0027 | 0.0016 | 0.0023 | 0.0024 | 2.62 | 0.63 | 0.98 | 0.02 | 0.53 | 1.00 | 0.00 |
| g_Veillonella | 0.0052 | 0.0062 | 0.0150 | 0.0118 | 0.0000 | 2.91 | 0.78 | 0.94 | 0.47 | 0.76 | 0.87 | 0.44 |
| g_Phascolaretobacterium | 0.0008 | 0.0017 | 0.0023 | 0.0037 | 0.0002 | 2.98 | 0.72 | 0.95 | 0.18 | 0.54 | 0.91 | 0.18 |
| g_Lachnospim | 0.0008 | 0.0017 | 0.0026 | 0.0036 | 0.0000 | 3.23 | 0.69 | 0.96 | 0.24 | 0.57 | 0.98 | 0.18 |
| g_Klebsiella | 0.0012 | 0.0023 | 0.0038 | 0.0038 | 0.0000 | 3.25 | 0.78 | 0.94 | 0.40 | 0.76 | 0.92 | 0.35 |
| g_Roseburia | 0.0007 | 0.0018 | 0.0033 | 0.0056 | 0.0000 | 4.54 | 0.71 | 0.94 | 0.26 | 0.56 | 0.96 | 0.29 |
| g_Parabacteroides | 0.0032 | 0.0053 | 0.0148 | 0.0114 | 0.0000 | 4.69 | 0.90 | 0.94 | 0.53 | 0.92 | 0.98 | 0.50 |
| g_Bacteroides | 0.0217 | 0.0229 | 0.1098 | 0.0441 | 0.0000 | 5.07 | 0.98 | 0.96 | 0.90 | 0.95 | 0.94 | 0.79 |
| g_Bifidobacterium | 0.0087 | 0.0104 | 0.0647 | 0.0324 | 0.0000 | 7.42 | 0.96 | 0.98 | 0.84 | 0.95 | 1.00 | 0.85 |
| g_Megamonas | 0.0004 | 0.0020 | 0.0041 | 0.037 | 0.3166 | 10.33 | 0.58 | 0.99 | 0.02 | 0.40 | 1.00 | 0.00 |
| g_Enterobacter | 0.0001 | 0.0003 | 0.0019 | 0.0021 | 0.0000 | 21.12 | 0.89 | 0.95 | 0.61 | 0.97 | 0.96 | 0.65 |

### Example 5. Breast Cancer Diagnostic Model Based on Metagenomic Analysis of Bacteria-Derived EVs Isolated from Urine

EVs were isolated from urine samples of 127 breast cancer patients and 220 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, sensitivity, and specificity, which are diagnostic performance indexes, were calculated by logistic regression analysis.

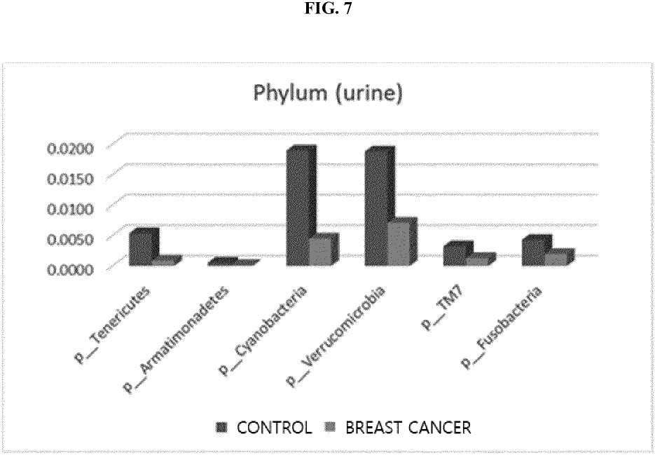

As a result of analyzing bacteria-derived EVs in urine at a phylum level, a diagnostic model developed using bacteria belonging to the phylum Tenericutes, the phylum Armatimonadetes, the phylum Cyanobacteria, the phylum Verrucomicrobia, the phylum TM7, and the phylum Fusobacteria as a biomarker exhibited significant diagnostic performance for breast cancer (see Table 7 and FIG. 7).

TABLE 7

| | Control | | Breast cancer | | t-test | | TrainingSet | | | stSet | | |
|---|---|---|---|---|---|---|---|---|---|---|---|---|
| | Mean | SD | Mean | SD | p-value | fold | AUC | sensitivity | specificity | AUC | sensitivity | specificity |
| p_Tenericates | 0.0053 | 0.0088 | 0.0008 | 0.0018 | 0.0000 | 0.16 | 0.81 | 0.79 | 0.63 | 0.78 | 0.88 | 0.50 |
| p_Armatimonadetes | 0.0005 | 0.0013 | 0.0001 | 0.0004 | 0.0000 | 0.22 | 0.72 | 0.83 | 0.42 | 0.71 | 0.84 | 0.45 |
| p_Cyanobacteria | 0.0189 | 0.0310 | 0.0005 | 0.0049 | 0.0000 | 0.24 | 0.78 | 0.77 | 0.60 | 0.74 | 0.87 | 0.47 |
| p_Vernicomierobia | 0.0187 | 0.0252 | 0.0071 | 0.0109 | 0.0000 | 0.38 | 0.76 | 0.82 | 0.50 | 0.76 | 0.82 | 0.53 |
| p_TM7 | 0.0032 | 0.0050 | 0.0013 | 0.0025 | 0.0000 | 0.40 | 0.74 | 0.88 | 0.54 | 0.75 | 0.88 | 0.45 |
| p_Fusobacteria | 0.0042 | 0.0062 | 0.0019 | 0.0037 | 0.0000 | 0.45 | 0.73 | 0.88 | 0.47 | 0.72 | 0.85 | 0.39 |

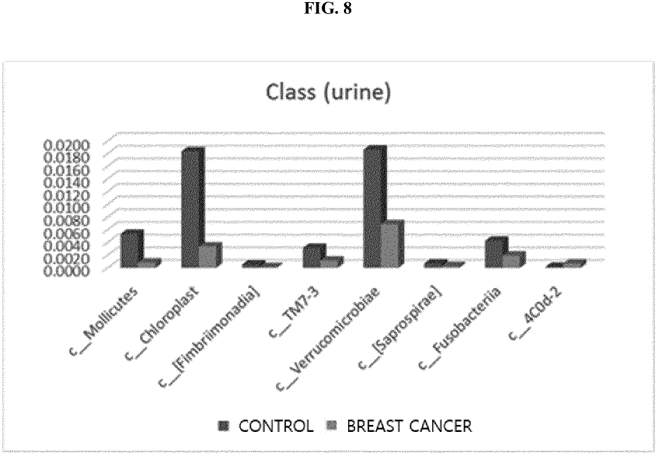

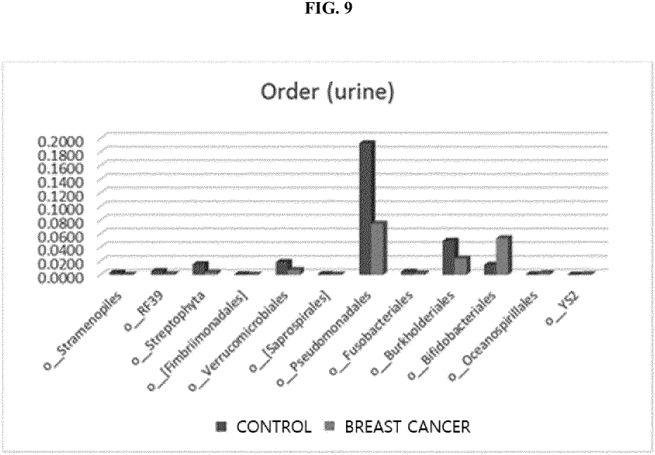

As a result of analyzing bacteria-derived EVs in urine at a class level, a diagnostic model developed using bacteria belonging to the class Mollicutes, the class Chloroplast, the class Fimbriimonadia, the class TM7-3, the class Verrucomicrobiae, the class Saprospirae, the class Fusobacteriia, and the class 4C0d-2 as a biomarker exhibited significant diagnostic performance for breast cancer (see Table 8 and FIG. 8).

TABLE 8

| | Control | | Breast cancer | | t-test | | Training set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| c_Mollicutes | 0.0053 | 0.0088 | 0.0008 | 0.0018 | 0.0000 | 0.15 | 0.81 | 0.79 | 0.64 | 0.7 8 | 0.88 | 0.50 |
| c_Chloroplast | 0.0183 | 0.0309 | 0.0034 | 0.0043 | 0.0000 | 0.18 | 0.80 | 0.76 | 0.66 | 0.77 | 0.87 | 0.55 |
| c_[Fimbriimonadia] | 0.0005 | 0.0013 | 0.0001 | 0.0004 | 0.000 0 | 0.22 | 0.72 | 0.83 | 0.42 | 0.71 | 0.84 | 0.42 |
| c_TM7-3 | 0.0031 | 0.00 50 | 0.0011 | 0.0024 | 0.0000 | 0.36 | 0.75 | 0.88 | 0.56 | 0.75 | 0.88 | 0.42 |
| c_Verrucomicrobiac | 0.0186 | 0.02 52 | 0.0069 | 0.0109 | 0.000 0 | 0.37 | 0.76 | 0.82 | 0.49 | 0.76 | 0.82 | 0.53 |
| c_[Saprospirae] | 0.0007 | 0.0018 | 0.0003 | 0.0009 | 0.0050 | 0.39 | 0.71 | 0.87 | 0.34 | 0.71 | 0.88 | 0.42 |
| c_Fusobacteriia | 0.0042 | 0.0062 | 0.0019 | 0.0037 | 0.0000 | 0.45 | 0.7 3 | 0.88 | 0.47 | 0.72 | 0.85 | 0.39 |
| c_4C0d-2 | 0.0002 | 0.0005 | 0.0007 | 0.0018 | 0.0022 | 4.06 | 0.72 | 0.84 | 0.38 | 0.75 | 0.90 | 0.47 |

As a result of analyzing bacteria-derived EVs in urine at order level, a diagnostic model developed using bacteria belonging to the order Stramenopiles, the order RF39, the order Streptophyta, the order Fimbriimonadales, the order Verrucomicrobiales, the order Saprospirales, the order Psuedomonadales, the order Fusobacteriales, the order Burkholderiales, the order Bifidobacteriales, the order Oceanospirillales, and the order YS2 as a biomarker exhibited significant diagnositic performance for breast cancer.

TABLE 9

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| o_Stramenopiles | 0.0029 | 0.0057 | 0.0000 | 0.0003 | 0.0000 | 0.01 | 0.78 | 0.75 | 0.66 | 0.79 | 0.84 | 0.53 |
| o_RF39 | 0.0052 | 0.0088 | 0.0008 | 0.0017 | 0.0000 | 0.14 | 0.81 | 0.79 | 0.66 | 0.79 | 0.88 | 0.50 |
| o_Streptophyta | 0.0154 | 0.0295 | 0.0033 | 0.0043 | 0.0000 | 0.22 | 0.77 | 0.77 | 0.58 | 0.74 | 0.85 | 0.45 |
| o_[Fimbriimonadales ] | 0.0005 | 0.0013 | 0.0001 | 0.0004 | 0.0000 | 0.22 | 0.72 | 0.83 | 0.42 | 0.71 | 0.84 | 0.42 |
| o_Verrueomicrobiales | 0.0186 | 0.0252 | 0.0069 | 0.0109 | 0.0000 | 0.37 | 0.76 | 0.82 | 0.49 | 0.76 | 0.82 | 0.53 |
| o_[Saprospirales] | 0.0007 | 0.0018 | 0.0003 | 0.0009 | 0.0050 | 0.39 | 0.71 | 0.87 | 0.34 | 0.71 | 0.88 | 0.42 |
| o_Pseudomonadales | 0.1925 | 0.1599 | 0.0748 | 0.0682 | 0.0000 | 0.39 | 0.80 | 0.82 | 0.60 | 0.79 | 0.84 | 0.47 |
| o_Fusobacteriales | 0.0042 | 0.0062 | 0.0019 | 0.003 7 | 0.0000 | 0.45 | 0.73 | 0.88 | 0.47 | 0.72 | 0.85 | 0.39 |
| o_Burkholderiales | 0.0495 | 0.1336 | 0.0236 | 0.0195 | 0.0051 | 0.48 | 0.71 | 0.87 | 0.39 | 0.70 | 0.88 | 0.45 |
| o_Bifidobacteriales | 0.0145 | 0.0214 | 0.0533 | 0.0424 | 0.0000 | 3.67 | 0.85 | 0.94 | 0.53 | 0.88 | 0.94 | 0.63 |
| o_Oceanospirillales | 0.0005 | 0.0023 | 0.0022 | 0.0045 | 0.0002 | 4.19 | 0.76 | 0.90 | 0.42 | 0.73 | 0.93 | 0.32 |
| o_YS2 | 0.0001 | 0.0004 | 0.0007 | 0.0018 | 0.0008 | 5.58 | 0.74 | 0.86 | 0.38 | 0.75 | 0.90 | 0.42 |

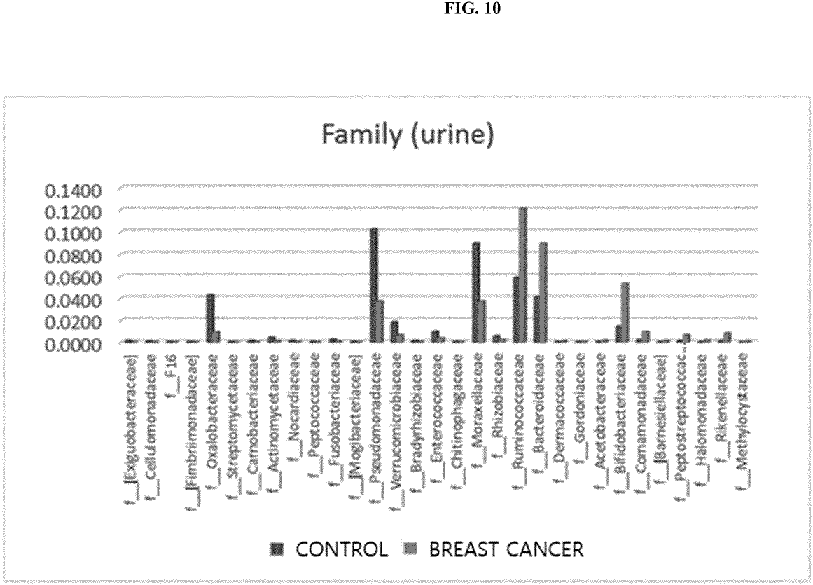

As a result of analyzing bacteria-derived EVs in urine at a family level, a diagnostic model developed using bacteria belonging to the family Exiguobacteraceae, the family Cellulomonadaceae, the family F 16, the family Fimbriimonadaceae, the family Oxalobacteraceae, the family Streptomycetaceae, the family Carnobacteriaceae, the family Actinomycetaceae, the family Nocardiaceae, the family Peptococcaceae, the family Fusobacteriaceae, the family Mogibacteriaceae, the family Pseudomonadaceae, the family Verrucomicrobiaceae, the family Bradyrhizobiaceae, the family Enterococcaceae, the family Chitinophagaceae, the family Moraxellaceae, the family Rhizobiaceae, the family Ruminococcaceae, the family Bacteroidaceae, the family Dermacoccaceae, the family Gordoniaceae, the family Acetobacteraceae, the family Bifidobacteriaceae, the family Comamonadaceae, the family Barnesiellaceae, the family Peptostreptococcaceae, the family Halomonadaceae, the family Rikenellaceae, and the family Methylocystaceae as a biomarker exhibited significant diagnostic performance for breast cancer (see Table 10 and FIG. 10).

TABLE 10

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| f_[Exiguobacleraceae] | 0.0016 | 0.0063 | 0.0001 | 0.0005 | 0.0004 | 0.05 | 0.75 | 0.81 | 0.49 | 0.71 | 0.81 | 0.47 |
| f_Cellulomonadaceae | 0.0011 | 0.0022 | 0.0002 | 0.0010 | 0.0000 | 0.19 | 0.77 | 0.88 | 0.47 | 0.71 | 0.84 | 0.42 |
| f_F16 | 0.0005 | 0.0017 | 0.0001 | 0.0006 | 0.0032 | 0.20 | 0.72 | 0.87 | 0.39 | 0.71 | 0.87 | 0.45 |
| f_[Fimbriimonadaceae] | 0.0005 | 0.0013 | 0.0001 | 0.0004 | 0.0000 | 0.22 | 0.72 | 0.83 | 0.42 | 0.71 | 0.84 | 0.42 |
| f_Oxalobacteraccae | 0.0431 | 0.1330 | 0.0095 | 0.0082 | 0.0002 | 0.22 | 0.74 | 0.84 | 0.51 | 0.71 | 0.88 | 0.45 |
| f_Streptomycetaceae | 0.0005 | 0.0015 | 0.0001 | 0.0006 | 0.0004 | 0.25 | 0.72 | 0.88 | 0.38 | 0.71 | 0.88 | 0.42 |
| f_Camobacteriaceae | 0.0014 | 0.0027 | 0.0004 | 0.0012 | 0.0000 | 0.26 | 0.73 | 0.88 | 0.43 | 0.76 | 0.84 | 0.50 |
| f_Actinomyeetaceae | 0.0047 | 0.0056 | 0.0013 | 0.0021 | 0.0000 | 0.27 | 0.76 | 0.80 | 0.51 | 0.83 | 0.85 | 0.45 |
| f_Nocanliaceae | 0.0016 | 0.0024 | 0.0005 | 0.0014 | 0.0000 | 0.32 | 0.77 | 0.84 | 0.52 | 0.73 | 0.84 | 0.42 |
| f_Peptocpccaceae | 0.0005 | 0.0011 | 0.0002 | 0.0008 | 0.0019 | 0.33 | 0.72 | 0.85 | 0.38 | 0.71 | 0.85 | 0.45 |
| f_Fusobacteriaceae | 0.0027 | 0.0048 | 0.0009 | 0.0024 | 0.0000 | 0.33 | 0.75 | 0.89 | 0.46 | 0.72 | 0.87 | 0.42 |
| f_[Mogibacteriaceae] | 0.0007 | 0.0012 | 0.0002 | 0.0006 | 0.0000 | 0.35 | 0.73 | 0.86 | 0.41 | 0.75 | 0.93 | 0.47 |
| f_Pseudomonadaceae | 0.1026 | 0.0897 | 0.0374 | 0.0441 | 0.0000 | 0.36 | 0.82 | 0.84 | 0.66 | 0.82 | 0.85 | 0.53 |
| f_Vemicomicrobiaceae | 0.0186 | 0.02 52 | 0.0069 | 0.0109 | 0.0000 | 0.37 | 0.76 | 0.82 | 0.49 | 0.76 | 0.82 | 0.53 |
| f_Bradyrhizobiaceae | 0.0015 | 0.0035 | 0.0006 | 0.0016 | 0.002 | 0.39 | 0.72 | 0.86 | 0.41 | 0.71 | 0.84 | 0.45 |
| f_Enerrocoeaaceae | 0.0097 | 0.0104 | 0.0039 | 0.0076 | 0.0000 | 0.40 | 0.77 | 0.86 | 0.51 | 0.77 | 0.85 | 0.47 |
| f_Chitinophagaceae | 0.0006 | 0.0013 | 0.0002 | 0.0009 | 0.0038 | 0.41 | 0.71 | 0.87 | 0.36 | 0.70 | 0.88 | 0.42 |
| f_Moraxellaceae | 0.0898 | 0.1050 | 0.0373 | 0.0361 | 0.0000 | 0.42 | 0.75 | 0.81 | 0.48 | 0.72 | 0.85 | 0.37 |
| f_Rhizobiaceae | 0.0059 | 0.0073 | 0.0025 | 0.0039 | 0.0000 | 0.42 | 0.76 | 0.82 | 0.53 | 0.75 | 0.90 | 0.45 |
| f_Ruminococeaceae | 0.0587 | 0.0460 | 0.1213 | 0.0793 | 0.0000 | 2.07 | 0.79 | 0.84 | 0.47 | 0.82 | 0.87 | 0.42 |
| f_Bacteroidaceae | 0.0415 | 0.0385 | 0.0896 | 0.0606 | 0.0000 | 2.16 | 0.80 | 0.86 | 0.48 | 0.83 | 0.90 | 0.50 |
| f_Dermacoecaecae | 0.0005 | 0.0015 | 0.0012 | 0.0023 | 0.0035 | 2.32 | 0.72 | 0.87 | 0.37 | 0.70 | 0.91 | 0.39 |
| f_Gordoniaecae | 0.0002 | 0.0007 | 0.0005 | 0.0012 | 0.0040 | 2.79 | 0.71 | 0.86 | 0.37 | 0.70 | 0.88 | 0.37 |
| f_Acetobacteraceae | 0.0006 | 0.0018 | 0.0018 | 0.0031 | 0.0001 | 2.98 | 0.72 | 0.87 | 0.33 | 0.73 | 0.93 | 0.42 |
| f_Bifidobacteriaceae | 0.0145 | 0.0214 | 0.0533 | 0.0424 | 0.0000 | 3.67 | 0.85 | 0.94 | 0.53 | 0.88 | 0.94 | 0.63 |
| f_Comamonadaceae | 0.0025 | 0.0039 | 0.0097 | 0.0138 | 0.0000 | 3.91 | 0.80 | 0.90 | 0.48 | 0.79 | 0.96 | 0.47 |
| f_[Barnesiellaceae] | 0.0004 | 0.0012 | 0.0015 | 0.0037 | 0.0012 | 4.07 | 0.73 | 0.89 | 0.36 | 0.71 | 0.90 | 0.42 |
| f_Peptostreptococcaceae | 0.0016 | 0.0044 | 0.0068 | 0.0213 | 0.0072 | 4.16 | 0.74 | 0.88 | 0.34 | 0.73 | 0.91 | 0.34 |
| f_Halomonadaceae | 0.0004 | 0.0022 | 0.0021 | 0.0045 | 0.0001 | 5.69 | 0.76 | 0.90 | 0.40 | 0.75 | 0.94 | 0.34 |
| f_Rikenellaeeae | 0.0014 | 0.0027 | 0.0083 | 0.0135 | 0.0000 | 5.98 | 0.84 | 0.90 | 0.58 | 0.79 | 0.93 | 0.42 |
| f_Methylocystacene | 0.0001 | 0.0007 | 0.0011 | 0.0025 | 0.0000 | 9.69 | 0.74 | 0.94 | 0.36 | 0.76 | 0.91 | 0.42 |

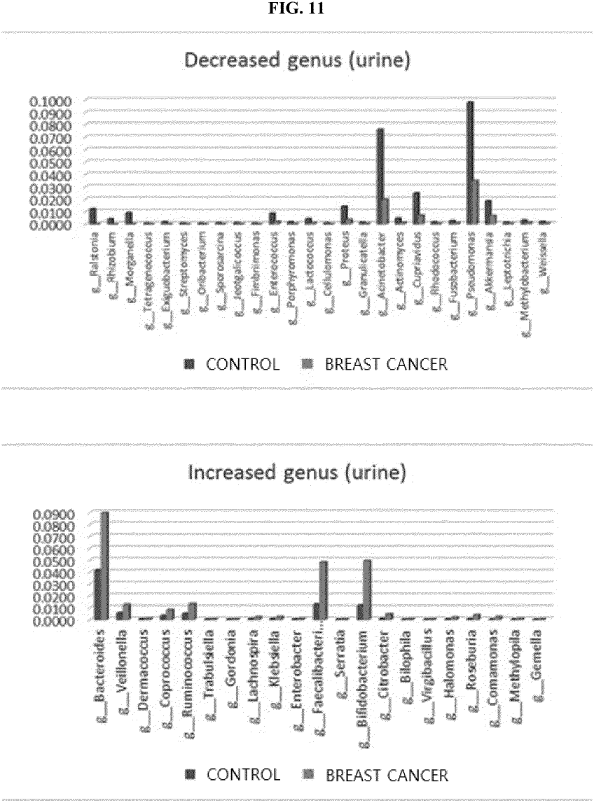

As a result of analyzing bacteria-derived EVs in urine at a genus level, a diagnostic model developed using bacteria belonging to the genus Ralstonia, the genus Rhizobium, the genus Morganella, the genus Tetragenococcus, the genus Exiguobacterium, the genus Streptomyces, the genus Oribacterium, the genus Sporosarcina, the genus Jeotgalicoccus, the genus Fimbriimonas, the genus Enterococcus, the genus Porphyromonas, the genus Lactococcus, the genus Cellulomonas, the genus Proteus, the genus Granulicatella, the genus Acinetobacter, the genus Actinomyces, the genus Cupriavidus, the genus Rhodococcus, the genus Fusobacterium, the genus Pseudomonas, the genus Akkermansia, the genus Leptotrichia, the genus Methylobacterium, the genus Weissella, the genus Bacteroides, the genus Veillonella, the genus Dermacoccus, the genus Coprococcus, the genus Ruminococcus, the genus Trabulsiella, the genus Gordonia, the genus Lachnospira, the genus Klebsiella, the genus Enterobacter, the genus Faecalibacterium, the genus Serratia, the genus Bifidobacterium, the genus Citrobacter, the genus Bilophila, the genus Virgibacillus, the genus Halomonas, the genus Roseburia, the genus Comamonas, the genus Methylopila, and the genus Gemella as a biomarker exhibited significant diagnostic performance for breast cancer (see Table 11 and FIG. 11).

TABLE 11

| | Control | | Breast cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | scensitivity | specificity |
| g_Ralstonia | 0.0122 | 0.0421 | 0.0001 | 0.0003 | 0.0000 | 0.00 | 0.81 | 0.80 | 0.64 | 0.86 | 0.90 | 0.53 |
| g_Rhizobium | 0.0041 | 0.0062 | 0.0000 | 0.0001 | 0.0000 | 0.01 | 0.96 | 0.88 | 0.98 | 0.97 | 0.85 | 0.95 |
| g_Monganella | 0.0090 | 0.0238 | 0.0002 | 0.0007 | 0.0000 | 0.02 | 0.79 | 0.83 | 0.59 | 0.76 | 0.84 | 0.47 |
| g_Tetragenococcus | 0.0006 | 0.0019 | 0.0000 | 0.0002 | 0.0000 | 0.03 | 0.74 | 0.86 | 0.39 | 0.73 | 0.88 | 0.45 |
| g_Exiguobucterium | 0.0016 | 0.0063 | 0.0001 | 0.0005 | 0.0004 | 0.05 | 0.75 | 0.81 | 0.49 | 0.71 | 0.81 | 0.47 |
| g_Sureptomyces | 0.0005 | 0.0015 | 0.0000 | 0.0003 | 0.0000 | 0.09 | 0.74 | 0.88 | 0.43 | 0.74 | 0.84 | 0.47 |
| g_Oribacteriam | 0.0005 | 0.0013 | 0.0001 | 0.0005 | 0.0000 | 0.13 | 0.74 | 0.87 | 0.38 | 0.73 | 0.87 | 0.42 |
| g_Sporosarcina | 0.0009 | 0.0022 | 0.0001 | 0.0005 | 0.0000 | 0.15 | 0.75 | 0.87 | 0.50 | 0.71 | 0.82 | 0.45 |
| g_Jeotgalicoecus | 0.0008 | 0.0019 | 0.0002 | 0.0009 | 0.0000 | 0.21 | 0.75 | 0.85 | 0.48 | 0.71 | 0.81 | 0.47 |
| g_Fimbriimonas | 0.0005 | 0.0013 | 0.0001 | 0.0004 | 0.0000 | 0.22 | 0.72 | 0.83 | 0.42 | 0.71 | 0.84 | 0.42 |
| g_Enterococeus | 0.0086 | 0.0097 | 0.0019 | 0.0041 | 0.0000 | 0.22 | 0.84 | 0.83 | 0.67 | 0.83 | 0.84 | 0.61 |
| g_Porphyromonas | 0.0015 | 0.0033 | 0.0003 | 0.0009 | 0.0000 | 0.23 | 0.73 | 0.85 | 0.47 | 0.72 | 0.85 | 0.42 |
| g_Lactococcus | 0.0043 | 0.0092 | 0.0010 | 0.0031 | 0.0000 | 0.23 | 0.75 | 0.84 | 0.48 | 0.78 | 0.85 | 0.47 |
| g_Cellulomonas | 0.0007 | 0.0017 | 0.0002 | 0.0010 | 0.0001 | 0.23 | 0.75 | 0.87 | 0.43 | 0.71 | 0.84 | 0.45 |
| g_Proteus | 0.0142 | 0.0236 | 0.0036 | 0.0354 | 0.0027 | 0.25 | 0.75 | 0.87 | 0.39 | 0.75 | 0.87 | 0.42 |
| g_Granulicatella | 0.0013 | 0.0026 | 0.0003 | 0.0011 | 0.0000 | 0.25 | 0.73 | 0.87 | 0.42 | 0.75 | 0.85 | 0.50 |
| g_Acinetobacter | 0.0761 | 0.1046 | 0.0198 | 0.0264 | 0.0000 | 0.26 | 0.77 | 0.78 | 0.50 | 0.76 | 0.81 | 0.50 |
| g_Actinomyces | 0.0047 | 0.0056 | 0.0013 | 0.0021 | 0.0000 | 0.27 | 0.76 | 0.81 | 0.52 | 0.83 | 0.88 | 0.45 |
| g_Cupriavidas | 0.0249 | 0.0865 | 0.0072 | 0.0065 | 0.0027 | 0.29 | 0.72 | 0.86 | 0.41 | 0.70 | 0.87 | 0.39 |
| g_Rhodococous | 0.0016 | 0.0024 | 0.0005 | 0.0013 | 0.0000 | 0.31 | 0.77 | 0.84 | 0.52 | 0.73 | 0.84 | 0.42 |
| g_Fusobacterium | 0.0026 | 0.0048 | 0.0009 | 0.0024 | 0.0000 | 0.33 | 0.74 | 0.89 | 0.44 | 0.72 | 0.87 | 0.42 |
| g_Pseudamonas | 0.0980 | 0.0879 | 0.0350 | 0.0420 | 0.0000 | 0.36 | 0.82 | 0.84 | 0.63 | 0.83 | 0.87 | 0.55 |
| g_Akkermansia | 0.0186 | 0.0251 | 0.0068 | 0.0109 | 0.0000 | 0.37 | 0.76 | 0.82 | 0.49 | 0.76 | 0.82 | 0.53 |
| g_Leptotrichia | 0.0012 | 0.0025 | 0.0005 | 0.0025 | 0.0058 | 0.38 | 0.71 | 0.88 | 0.38 | 0.72 | 0.87 | 0.42 |
| g_Methylobacterium | 0.0032 | 0.0051 | 0.0013 | 0.0033 | 0.0000 | 0.42 | 0.74 | 0.90 | 0.48 | 0.71 | 0.85 | 0.42 |
| g_Weissella | 0.0017 | 0.0035 | 0.0008 | 0.0020 | 0.0014 | 0.46 | 0.73 | 0.89 | 0.48 | 0.68 | 0.84 | 0.34 |
| g_Baeteroides | 0.0415 | 0.0385 | 0.0895 | 0.0606 | 0.0000 | 2.16 | 0.80 | 0.86 | 0.48 | 0.83 | 0.90 | 0.50 |
| g_Veillonella | 0.0057 | 0.0071 | 0.0126 | 0.0176 | 0.0000 | 2.22 | 0.75 | 0.88 | 0.39 | 0.72 | 0.00 | 0.37 |
| g_Dermacoccus | 0.0005 | 0.0015 | 0.0012 | 0.0023 | 0.0035 | 2.32 | 0.72 | 0.87 | 0.37 | 0.70 | 0.91 | 0.39 |
| g_Coprococeus | 0.0034 | 0.0041 | 0.0082 | 0.0116 | 0.0000 | 2.42 | 0.74 | 0.88 | 0.47 | 0.75 | 0.90 | 0.42 |
| g_Rurninococcus | 0.0051 | 0.0053 | 0.0132 | 0.0235 | 0.0002 | 2.61 | 0.74 | 0.85 | 0.38 | 0.73 | 0.90 | 0.32 |
| g_Trabulsiell | 0.0002 | 0.0010 | 0.0005 | 0.0009 | 0.0024 | 2.61 | 0.75 | 0.87 | 0.40 | 0.74 | 0.84 | 0.39 |
| g_Gordonia | 0.0002 | 0.0007 | 0.0005 | 0.0012 | 0.0040 | 2.79 | 0.71 | 0.86 | 0.37 | 0.70 | 0.88 | 0.37 |
| g_Lachnospira | 0.0008 | 0.0017 | 0.0023 | 0.0030 | 0.0000 | 2.85 | 0.76 | 0.86 | 0.42 | 0.71 | 0.81 | 0.39 |
| g_Klebsiella | 0.0008 | 0.0015 | 0.0023 | 0.0031 | 0.0000 | 2.98 | 0.73 | 0.88 | 0.41 | 0.78 | 0.93 | 0.45 |
| g_Enterobacter | 0.0003 | 0.0009 | 0.0010 | 0.0015 | 0.0000 | 3.69 | 0.74 | 0.90 | 0.39 | 0.79 | 0.88 | 0.50 |
| g_Faecalibacterium | 0.0130 | 0.0144 | 0.0483 | 0.0427 | 0.0000 | 3.73 | 0.88 | 0.91 | 0.70 | 0.90 | 0.90 | 0.68 |
| g_Serraria | 0.0001 | 0.0003 | 0.0004 | 0.0009 | 0.0003 | 3.88 | 0.72 | 0.87 | 0.34 | 0.72 | 0.91 | 0.42 |
| g_Bifidobaclerium | 0.0121 | 0.0122 | 0.0494 | 0.0417 | 0.0000 | 4.09 | 0.85 | 0.91 | 0.56 | 0.88 | 0.90 | 0.68 |
| g_Citrobacter | 0.0010 | 0.0035 | 0.0046 | 0.0077 | 0.0000 | 4.70 | 0.79 | 0.93 | 0.51 | 0.79 | 0.94 | 0.42 |
| g_Bilophila | 0.0001 | 0.0004 | 0.0006 | 0.0018 | 0.0032 | 4.75 | 0.74 | 0.86 | 0.37 | 0.73 | 0.87 | 0.42 |
| g_Virgibcillus | 0.0001 | 0.0005 | 0.0004 | 0.0012 | 0.0038 | 5.16 | 0.75 | 0.86 | 0.39 | 0.70 | 0.91 | 0.42 |
| g_Halomonas | 0.0003 | 0.0021 | 0.0020 | 0.0045 | 0.0001 | 6.14 | 0.76 | 0.90 | 0.40 | 0.75 | 0.94 | 0.37 |
| g_Roseburia | 0.0006 | 0.0012 | 0.0039 | 0.0127 | 0.0045 | 6.24 | 0.78 | 0.88 | 0.49 | 0.77 | 0.90 | 0.37 |
| g_Comamonas | 0.0002 | 0.0005 | 0.0024 | 0.0047 | 0.0000 | 15.21 | 0.80 | 0.90 | 0.51 | 0.85 | 0.97 | 0.50 |
| g_Methylopila | 0.0001 | 0.0005 | 0.0011 | 0.0025 | 0.0000 | 15.37 | 0.74 | 0.94 | 0.34 | 0.76 | 0.91 | 0.42 |
| g_Gemella | 0.0000 | 0.0002 | 0.0005 | 0.0012 | 0.0000 | 16.91 | 0.76 | 0.88 | 0.40 | 0.78 | 0.91 | 0.50 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

According to the present invention, a risk of developing breast cancer may be predicted through metagenomic analysis of bacteria-derived extracellular vesicles by using a human body-derived sample, and thus the onset of breast cancer may be delayed or breast cancer may be prevented through appropriate management by early diagnosis and prediction of a risk group for breast cancer, and, even after breast cancer occurs, early diagnosis for breast cancer may be implemented, thereby lowering the incidence rate of breast cancer and increasing therapeutic effects. In addition, patients diagnosed with breast cancer are able to avoid exposure to causative factors predicted by metagenomic analysis, whereby the progression of cancer may be ameliorated, or recurrence of breast cancer may be prevented.

1. A method of providing information for breast cancer diagnosis, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles of the subject sample with that of a normal individual-derived sample through sequencing of a product of the PCR.

2. The method of claim 1, wherein the comparing in step (c) comprises comparing an increase or decrease of content of extracellular vesicles derived from one or more bacteria selected from the group consisting of the phylum Fusobacteria, Bacteroidetes, the phylum Tenericutes, the phylum Armatimonadetes, the phylum Cyanobacteria, the phylum Verrucomicrobia, and the phylum TM7.

3. The method of claim 1, wherein the comparing in step (c) comprises comparing an increase or decrease of content of extracellular vesicles derived from one or more bacteria selected from the group consisting of the class Fusobacteriia, the class Alphaproteobacteria, the class Chloroplast, the class TM7-3, the class Bacteroidia, the class Mollicutes, the class Fimbriimonadia, the class Verrucomicrobiae, the class Saprospirae, and the class 4C0d-2.

4. The method of claim 1, wherein the comparing in step (c) comprises comparing an increase or decrease of content of extracellular vesicles derived from one or more bacteria selected from the group consisting of the order Fusobacteriales, the order Sphingomonadales, the order Neisseriales, the order Rhizobiales, the order Rhodospirillales, the order Actinomycetales, the order Bacillales, the order Streptophyta, the order Caulobacterales, the order Pseudomonadales, the order Bacteroidales, the order Enterobacteriales, the order Bifidobacteriales, the order Stramenopiles, the order RF39, the order Fimbriimonadales, the order Verrucomicrobiales, the order Saprospirales, the order Burkholderiales, the order Bifidobacteriales, the order Oceanospirillales, and the order YS2.

5. The method of claim 1, wherein the comparing in step (c) comprises comparing an increase or decrease in content of extracellular vesicles derived from one or more bacteria selected from the group consisting of the family Staphylococcaceae, the family Fusobacteriaceae, the family Actinomycetaceae, the family Brevibacteriaceae, the family Peptostreptococcaceae, the family Rhizobiaceae, the family Corynebacteriaceae, the family Methylobacteriaceae, the family Propionibacteriaceae, the family Sphingomonadaceae, the family Neisseriaceae, the family Flavobacteriaceae, the family Intrasporangiaceae, the family Nocardiaceae, the family Caulobacteraceae, the family Micrococcaceae, the family Oxalobacteraceae, the family Moraxellaceae, the family Pseudomonadaceae, the family Porphyromonadaceae, the family Clostridiaceae, the family Rikenellaceae, the family Veillonellaceae, the family Enterobacteriaceae, the family S24-7, the family Bacteroidaceae, the family Bifidobacteriaceae, the family Exiguobacteraceae, the family Cellulomonadaceae, the family F16, the family Fimbriimonadaceae, the family Streptomycetaceae, the family Carnobacteriaceae, the family Peptococcaceae, the family Mogibacteriaceae, the family Verrucomicrobiaceae, the family Bradyrhizobiaceae, the family Enterococcaceae, the family Chitinophagaceae, the family Rhizobiaceae, the family Ruminococcaceae, the family Dermacoccaceae, the family Gordoniaceae, the family Acetobacteraceae, the family Comamonadaceae, the family Barnesiellaceae, the family Halomonadaceae, and the family Methylocystaceae.

6. The method of claim 1, wherein the comparing in step (c) comprises comparing an increase or decrease in content of extracellular vesicles derived from one or more bacteria selected from the group consisting of the genus Hydrogenophilus, the genus Staphylococcus, the genus Fusobacterium, the genus Actinomyces, the genus Brevibacterium, the genus Granulicatella, the genus Neisseria, the genus Rothia, the genus Corynebacterium, the genus Brevundimonas, the genus Propionibacterium, the genus Porphyromonas, the genus Sphingomonas, the genus Methylobacterium, the genus Micrococcus, the genus Coprococcus, the genus Rhodococcus, the genus Cupriavidus, the genus Acinetobacter, the genus Pseudomonas, the genus Enhydrobacter, the genus Ruminococcus, the genus Dialister, the genus Tepidimonas, the genus Veillonella, the genus Phascolarctobacterium, the genus Lachnospira, the genus Klebsiella, the genus Roseburia, the genus Parabacteroides, the genus Bacteroides, the genus Bifidobacterium, the genus Megamonas, the genus Enterobacter, the genus Rhizobium, the genus Morganella, the genus Tetragenococcus, the genus Streptomyces, the genus Oribacterium, the genus Sporosarcina, the genus , the genus Fimbriimonas, the genus Enterococcus, the genus Lactococcus, the genus Cellulomonas, the genus Proteus, the genus Akkermansia, the genus Leptotrichia, the genus Weissella, the genus Veillonella, the genus Dermacoccus, the genus Trabulsiella, the genus Gordonia, the genus Faecalibacterium, the genus Serratia, the genus Citrobacter, the genus Bilophila, the genus Virgibacillus, the genus Halomonas, the genus Comamonas, the genus Methylopila, and the genus Gemella.

7. The method of claim 1, wherein the subject sample is blood or urine.

8. The method of claim 7, wherein the blood is whole blood, serum, plasma, or blood mononuclear cells.

9. A method of diagnosing breast cancer, the method comprising:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles of the subject sample with that of a normal individual-derived sample through sequencing of a product of the PCR.

10. The method of claim 9, wherein the comparing in step (c) comprises comparing an increase or decrease in content of extracellular vesicles derived from one or more bacteria selected from the group consisting of the phylum Fusobacteria, the phylum Bacteroidetes, the phylum Tenericutes, the phylum Armatimonadetes, the phylum Cyanobacteria, the phylum Verrucomicrobia, and the phylum TM7.

11. The method of claim 9, wherein the comparing in step (c) comprises comparing an increase or decrease in content of extracellular vesicles derived from one or more bacteria selected from the group consisting of the class Fusobacteriia, the class Alphaproteobacteria, the class Chloroplast, the class TM7-3, the class Bacteroidia, the class Mollicutes, the class Fimbriimonadia, the class Verrucomicrobiae, the class Saprospirae, and the class 4C0d-2.

12. The method of claim 9, wherein the comparing in step (c) comprises comparing an increase or decrease in content of extracellular vesicles derived from one or more bacteria selected from the group consisting of the order Fusobacteriales, the order Sphingomonadales, the order Neisseriales, the order Rhizobiales, the order Rhodospirillales, the order Actinomycetales, the order Bacillales, the order Streptophyta, the order Caulobacterales, the order Pseudomonadales, the order Bacteroidales, the order Enterobacteriales, the order Bifidobacteriales, the order Stramenopiles, the order RF39, the order Fimbriimonadales, the order Verrucomicrobiales, the order Saprospirales, the order Burkholderiales, the order Bifidobacteriales, the order Oceanospirillales, and the order YS2.

13. The method of claim 9, wherein the comparing in step (c) comprises comparing an increase or decrease in content of extracellular vesicles derived from one or more bacteria selected from the group consisting of the family Staphylococcaceae, the family Fusobacteriaceae, the family Actinomycetaceae, the family Brevibacteriaceae, the family Peptostreptococcaceae, the family Rhizobiaceae, the family Corynebacteriaceae, the family Methylobacteriaceae, the family Propionibacteriaceae, the family Sphingomonadaceae, the family Neisseriaceae, the family Flavobacteriaceae, the family Intrasporangiaceae, the family Nocardiaceae, the family Caulobacteraceae, the family Micrococcaceae, the family Oxalobacteraceae, the family Moraxellaceae, the family Pseudomonadaceae, the family Porphyromonadaceae, the family Clostridiaceae, the family Rikenellaceae, the family Veillonellaceae, the family Enterobacteriaceae, the family S24-7, the family Bacteroidaceae, the family Bifidobacteriaceae, the family Exiguobacteraceae, the family Cellulomonadaceae, the family F16, the family Fimbriimonadaceae, the family Streptomycetaceae, the family Carnobacteriaceae, the family Peptococcaceae, the family Mogibacteriaceae, the family Verrucomicrobiaceae, the family Bradyrhizobiaceae, the family Enterococcaceae, the family Chitinophagaceae, the family Rhizobiaceae, the family Ruminococcaceae, the family Dermacoccaceae, the family Gordoniaceae, the family Acetobacteraceae, the family Comamonadaceae, the family Barnesiellaceae, the family Halomonadaceae, and the family Methylocystaceae.

14. The method of claim 9, wherein the comparing in step (c) comprises comparing an increase or decrease in content of extracellular vesicles derived from one or more bacteria selected from the group consisting of the genus Hydrogenophilus, the genus Staphylococcus, the genus Fusobacterium, the genus Actinomyces, the genus Brevibacterium, the genus Granulicatella, the genus Neisseria, the genus Rothia, the genus Corynebacterium, the genus Brevundimonas, the genus Propionibacterium, the genus Porphyromonas, the genus Sphingomonas, the genus Methylobacterium, the genus Micrococcus, the genus Coprococcus, the genus Rhodococcus, the genus Cupriavidus, the genus Acinetobacter, the genus Pseudomonas, the genus Enhydrobacter, the genus Ruminococcus, the genus Dialister, the genus Tepidimonas, the genus Veillonella, the genus Phascolarctobacterium, the genus Lachnospira, the genus Klebsiella, the genus Roseburia, the genus Parabacteroides, the genus Bacteroides, the genus Bifidobacterium, the genus Megamonas, the genus Enterobacter, the genus Ralstonia, the genus Rhizobium, the genus Morganella, the genus Tetragenococcus, the genus Exiguobacterium, the genus Streptomyces, the genus Oribacterium, the genus Sporosarcina, the genus Jeotgalicoccus, the genus Fimbriimonas, the genus Enterococcus, the genus Lactococcus, the genus Cellulomonas, the genus Proteus, the genus Akkermansia, the genus Leptotrichia, the genus Weissella, the genus Veillonella, the genus Dermacoccus, the genus Trabulsiella, the genus Gordonia, the genus Faecalibacterium, the genus Serratia, the genus Citrobacter, the genus Bilophila, the genus Virgibacillus, the genus Halomonas, the genus Comamonas, the genus Methylopila, and the genus Gemella.

15. The method of claim 9, wherein the subject sample is blood or urine.

16. The method of claim 15, wherein the blood is whole blood, serum, plasma, or blood mononuclear cells.
